(19) United States
(12) Patent Application Publication
(10) Pub. No.: US 2026/0258184 A1
(43) Pub. Date: Sep. 3, 2026

(54) ASYMMETRIC BIS(VINYLBENZYL) FLUORENE, THERMOSETTING HYDROCARBON RESIN, THERMOSETTING CROSS-LINKED RESIN, AND PREPARATION METHODS AND USE THEREOF

(71) Applicant: Shandong Xingshun New Material INC., Heze City (CN)

(72) Inventors: Xiaoxing GU, Heze City (CN); Guoyuan LU, Heze City (CN)

(21) Appl. No.: 19/235,550

(22) Filed: Jun. 11, 2025

(30) Foreign Application Priority Data
Feb. 25, 2025 (CN) 202510212948.1

(51) Int. Cl. C08F 290/06; C07C 13/567

(52) U.S. Cl. CPC C08F 290/062; C07C 13/567; C08F 2810/20

(57) ABSTRACT

Provided are an asymmetric bis(vinylbenzyl) fluorene (9-(2-vinylbenzyl)-9′-(4-vinylbenzyl)-9H-fluorene, o,p-BVBF), and a thermosetting hydrocarbon resin, preparation methods, and use thereof, as well as a thermosetting hydrocarbon resin of the o,p-BVBF and a symmetric bis(vinylbenzyl) fluorene (9,9′-bis(4-vinylbenzyl)-9H-fluorene, p,p-BVBF), and a thermosetting cross-linked resin of the o,p-BVBF and/or the p,p-BVBF as cross-linking agent(s) and alkenyl-terminated polyphenylene ether, and preparation methods and use thereof.

### CROSS REFERENCE TO RELATED APPLICATION

This application claims the benefit of and priority to Chinese Patent Application No. 202510212948.1, filed Feb. 25, 2025, which is hereby incorporated by reference herein in its entirety.

### TECHNICAL FIELD

The present disclosure pertains to the technical field of high-frequency and high-speed substrate materials, and specifically relates to an asymmetric bis(vinylbenzyl) fluorene, a thermosetting hydrocarbon resin, a thermosetting cross-linked modified polyphenylene ether resin, and preparation methods and use thereof.

### BACKGROUND

With the rapid development of 5G/6G communication, artificial intelligence, augmented reality/virtual reality (AR/VR), satellite navigation and autonomous driving, electronic products require high frequency, high speed, and larger capacity to transmit sound, video, and data. Further, the electronic products tend to develop in the direction of lightweight, thin, and small. In order to maintain higher transmission rates and signal integrity, laminated resin materials in core components (i.e., printed circuit board, PCB) of the electronic products are required to have lower dielectric constant, lower dielectric dissipation factor, higher glass transition temperature, and greater peel strength. Therefore, the key technical problem that urgently needs to be solved lies in further improving the dielectric and thermomechanical properties of resin materials.

Epoxy resin, due to its low cost, is the main resin material used for printed circuit boards. However, its relatively high dielectric constant and high dielectric dissipation factor make it difficult to achieve a suitable low dielectric dissipation factor in terms of high-frequency signals, thereby making it difficult to meet the requirements of high-speed signal transmission. Fluororesins represented by polytetrafluoroethylene have the properties of low dielectric constant and low dielectric dissipation factor, but the fluororesins are thermoplastic resins that are difficult to realize the preparation of multi-layer, lightweight, thin, and small printed circuit boards by processing and molding. Low-molecular-weight modified polyphenylene oxide (mPPO) terminated with vinylbenzyl or acryloyl has lower dielectric constant and dielectric dissipation factor. mPPO cross-linked resin formed from mPPO and diene (such as 1,2-bis(4-vinylphenyl) ethane, BVPE) has better thermal and mechanical properties and has been used as a laminated resin material for high-end printed circuit boards (PCBs). [0005]9,9-Bisubstituted fluorenylhydrocarbon derivatives, due to their unique Cardo structure, exhibit good photoelectric properties such as low dielectric constant and low dielectric dissipation factor, high refractive index and transparency, as well as good heat resistance, moisture resistance, and good solubility in organic solvents. The 9,9-bisubstituted fluorenylhydrocarbon derivatives are widely used as materials for microelectronic devices.

Japanese patent publication JP2003283076A discloses a mixture prepared by reacting vinyl benzyl chloride (a mixture of meta and para isomers at a mass ratio of 1:1) and allyl chloride with fluorene. A thermosetting resin formed therefrom exhibits a dielectric constant (5 GHz) of 4.0 and a dielectric dissipation factor (5 GHz) of 0.0035, which are obviously too high. Also, the presence of an allyl group on a 9-fluorenyl group results in a lower glass transition temperature of a composite. Patent publication WO2022207741A1 discloses a thermosetting resin formed from a mixture of vinylbenzylindene and 9,9-divinylbenzyl-9H-fluorene containing meta isomer, with a dielectric dissipation factor (10 GHz) of 0.00089. The dielectric constant and the dielectric dissipation factor are still relatively high, when using as a resin for high-frequency and high-speed substrates. Further, in patent publication WO2022207741A1, bismaleimide is used as a cross-linking agent to increase the glass transition temperature. Although the bismaleimide contains polar groups, and could improve the glass transition temperature, it would lead to an increase in the dielectric dissipation factor. CN1501899A discloses reacting a mixture of vinyl benzyl chloride (meta and para isomers at a mass ratio of 1:1) with fluorene to prepare a mixture of 9,9-bis(vinylbenzyl)-9H-fluorene meta and para isomers having a melting point of 142° C. A thermosetting resin formed therefrom has a dielectric constant and a dielectric dissipation factor (1 MHz) of 0.0013; the dielectric dissipation factor is obviously high, and thus the thermosetting resin is not suitable as a resin material for the high-frequency and high-speed substrates.

In order to meet the requirements of high-frequency and high-speed transmission of the new generation of printed circuit laminates, the key technical problems that urgently need to be solved are to further reduce the dielectric constant and the dielectric dissipation factor, and further improve the glass transition temperature of a resin material in the printed circuit boards.

### SUMMARY

In view of this, the present disclosure provides an asymmetric bis(vinylbenzyl) fluorene, a thermosetting hydrocarbon resin prepared by using the asymmetric bis(vinylbenzyl) fluorene as a raw material, and a preparation method and use thereof, a thermosetting hydrocarbon resin prepared by using the asymmetric bis(vinylbenzyl) fluorene and/or a symmetric bis(vinylbenzyl) fluorene as raw materials, and a preparation method and use thereof, also a thermosetting cross-linked polyphenylene ether resin cross-linked with alkenyl-terminated polyphenylene ether prepared by using the asymmetric bis(vinylbenzyl) fluorene, the symmetric bis(vinylbenzyl) fluorene, or a mixture of both as cross-linking agents, and a preparation method and use thereof. Due to the fact that: the asymmetric bis(vinylbenzyl) fluorene (9-(2-vinylbenzyl)-9′-(4-vinylbenzyl)-9H-fluorene, o,p-BVBF) according to the present disclosure is a non-polar hydrocarbon compound, and two asymmetric substituents of a 9-position fluorene group constitute a rigid cardo structure, the thermosetting hydrocarbon resin and the thermosetting cross-linked polyphenylene ether resin exhibit small dielectric dissipation factor, low dielectric constant, and high glass transition temperature. Therefore, the thermosetting hydrocarbon resin and the thermosetting cross-linked polyphenylene ether resin have great application prospects as resin materials for high-frequency and high-speed substrates.

In order to solve the above technical problems, the present disclosure provides an asymmetric bis(vinylbenzyl) fluorene, where the asymmetric bis(vinylbenzyl) fluorene is 9-(2-vinylbenzyl)-9′-(4-vinylbenzyl)-9H-fluorene having a structure shown in formula 1:

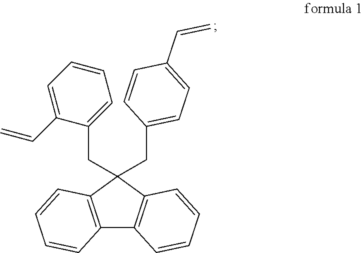 [SMILES: C=Cc1ccc(CC2(Cc3ccccc3C=C)c3ccccc3-c3ccccc32)cc1]

and
the 9-(2-vinylbenzyl)-9′-(4-vinylbenzyl)-9H-fluorene exhibits a thermal analysis melting endothermic peak in a temperature range of 169-175° C., and has a monoclinic crystal system and a space group of P2₁/c.

The present disclosure provides a method for preparing the asymmetric bis(vinylbenzyl) fluorene as described in above technical solutions, including:
mixing fluorene, a basic reagent, a polymerization inhibitor, and a polar aprotic solvent to obtain a fluorene-containing mixed system; and
dropwise adding 2-vinylbenzyl chloride and 4-vinylbenzyl chloride sequentially to the fluorene-containing mixed system, or dropwise adding a mixed solution of 2-vinylbenzyl chloride and 4-vinylbenzyl chloride to the fluorene-containing mixed system, and subjecting a resulting mixture to nucleophilic substitution reaction to obtain the asymmetric bis(vinylbenzyl) fluorene.

In some embodiments, the basic reagent includes at least one selected from the group consisting of an alkali metal hydroxide, an alkali metal alkoxide, sodium hydride, and potassium hydride;
the polymerization inhibitor includes at least one selected from the group consisting of nitromethane, nitrobenzene, o-nitrophenol, phenothiazine, 2-phenylnaphthylamine, hydroquinone, catechol, p-methoxyphenol, 2,6-di-tert-butyl-p-cresol, p-tert-butyl catechol, 2,2,6,6-tetramethylpiperidine oxynitride, and 2,2,6,6-tetramethyl-4-hydroxypiperidine oxynitride;
the polar aprotic solvent includes one selected from the group consisting of dimethyl sulfoxide, sulfolane, N,N-dimethylformamide, N,N-diethylformamide, acetonitrile, and hexamethylphosphoramide;
a molar ratio of the fluorene to the basic reagent is in a range of 1:1.8 to 1:5;
a molar ratio of the 2-vinylbenzyl chloride to the 4-vinylbenzyl chloride is in a range of 2-6:4-8; and
a ratio of a total amount in moles of the 2-vinylbenzyl chloride and the 4-vinylbenzyl chloride to an amount in moles of the fluorene is in a range of 1.8:1 to 2.2:1.

In some embodiments, the nucleophilic substitution reaction is conducted at a temperature of 20-50° C. for 2-8 hours.

The present disclosure provides a method for preparing the asymmetric bis(vinylbenzyl) fluorene as described in above technical solutions, including:
mixing fluorene, a basic reagent, a polymerization inhibitor, a phase transfer catalyst, and a solvent to obtain a fluorene-containing phase transfer catalytic system; and
dropwise adding 2-vinylbenzyl chloride and 4-vinylbenzyl chloride sequentially to the fluorene-containing phase transfer catalytic system, or dropwise adding a mixed solution of 2-vinylbenzyl chloride and 4-vinylbenzyl chloride to the fluorene-containing phase transfer catalytic system, and subjecting a resulting mixture to nucleophilic substitution reaction under the phase transfer catalysis conditions to obtain the asymmetric bis(vinylbenzyl) fluorene.

In some embodiments, the basic reagent includes at least one selected from the group consisting of an alkali metal hydroxide and an alkali metal alkoxide;
the polymerization inhibitor includes at least one selected from the group consisting of nitromethane, nitrobenzene, o-nitrophenol, phenothiazine, 2-phenylnaphthylamine, hydroquinone, catechol, p-methoxyphenol, 2,6-di-tert-butyl-p-cresol, p-tert-butyl catechol, 2,2,6,6-tetramethylpiperidine oxynitride, and 2,2,6,6-tetramethyl-4-hydroxypiperidine oxynitride;
the phase transfer catalyst includes at least one selected from the group consisting of a quaternary ammonium salt, a quaternary phosphonium salt, and polyethylene glycol;
a mass of the phase transfer catalyst is 5-25% of a mass of the fluorene;
a molar ratio of the fluorene to the basic reagent is in a range of 1:1.8 to 1:5;
a molar ratio of the 2-vinylbenzyl chloride to the 4-vinylbenzyl chloride is in a range of 2-6:4-8; and
a ratio of a total amount in moles of the 2-vinylbenzyl chloride and the 4-vinylbenzyl chloride to an amount in moles of the fluorene is in a range of 1.8:1 to 2.2:1.

In some embodiments, the nucleophilic substitution reaction under phase transfer catalysis conditions is conducted at a temperature of 25-75° C. for 8-18 hours.

The present disclosure provides a thermosetting resin, including at least one selected from the group consisting of a thermosetting hydrocarbon resin and a thermosetting cross-linked resin, where the thermosetting hydrocarbon resin includes at least one selected from the group consisting of a thermosetting hydrocarbon resin prepared by using 9-(2-vinylbenzyl)-9′-(4-vinylbenzyl)-9H-fluorene as a raw material and a thermosetting hydrocarbon resin prepared by using 9-(2-vinylbenzyl)-9′-(4-vinylbenzyl)-9H-fluorene and 9,9′-bis(4-vinylbenzyl)-9H-fluorene as raw materials;
the thermosetting cross-linked resin includes a thermosetting cross-linked resin prepared by using a cross-linking agent and an alkenyl-terminated polyphenylene ether as raw materials, the cross-linking agent includes at least one selected from the group consisting of 9-(2-vinylbenzyl)-9′-(4-vinylbenzyl)-9H-fluorene and 9,9′-bis(4-vinylbenzyl)-9H-fluorene, and the alkenyl-terminated polyphenylene ether has a structure selected from the group consisting of formula 2, formula 3, formula 4, and formula 5:

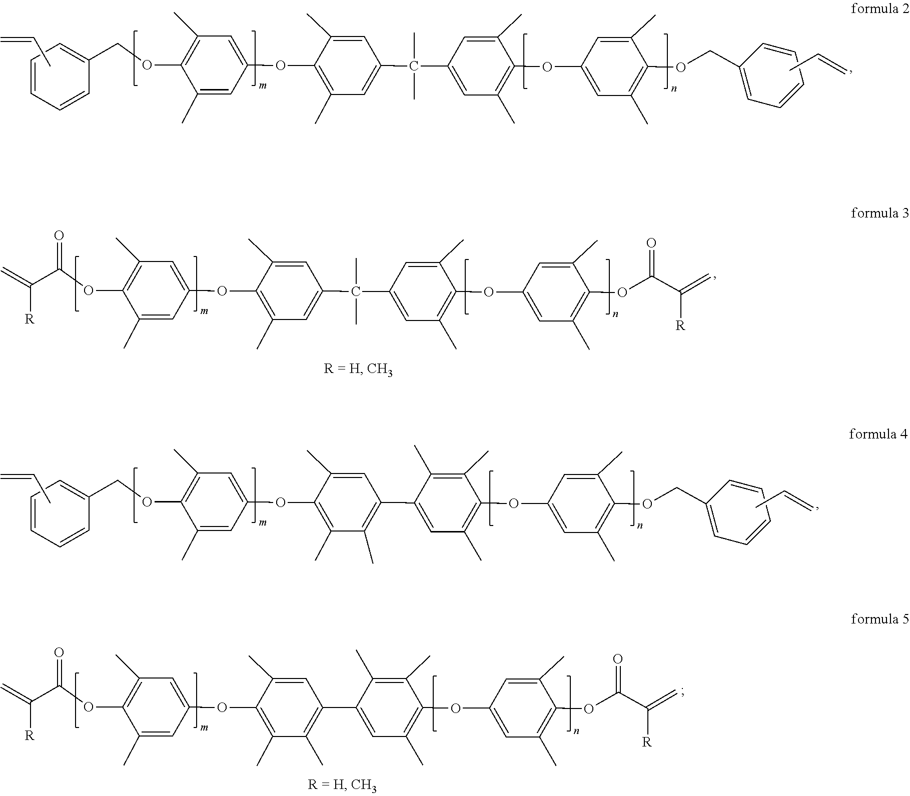

and
the 9-(2-vinylbenzyl)-9′-(4-vinylbenzyl)-9H-fluorene is the asymmetric bis(vinylbenzyl) fluorene as described in above technical solutions, or the asymmetric bis(vinylbenzyl) fluorene prepared by the method as described in above technical solutions.

The present disclosure provides a method for preparing the thermosetting resin as described in above technical solutions, including:
dissolving the raw material(s) and an initiator in toluene to obtain a mixed solution; and
removing the toluene from the mixed solution, and then conducting melting thermal curing to obtain the thermosetting resin.

The present disclosure provides use of the thermosetting resin as described in above technical solutions or the thermosetting resin prepared by the method as described in above technical solutions as a resin material for a high-frequency and high-speed substrate.

The present disclosure provides an asymmetric bis(vinylbenzyl) fluorene, having a structure shown in formula 1:

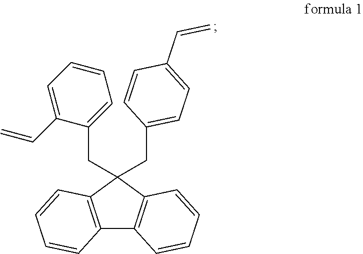 [SMILES: C=Cc1ccc(CC2(Cc3ccccc3C=C)c3ccccc3-c3ccccc32)cc1]

and
the 9-(2-vinylbenzyl)-9′-(4-vinylbenzyl)-9H-fluorene exhibits a thermal analysis melting endothermic peak in a temperature range of 169-175° C., and has a monoclinic crystal system and a space group of P2₁/c. The bis(vinylbenzyl) fluorene (9-(2-vinylbenzyl)-9′-(4-vinylbenzyl)-9H-fluorene (o,p-BVBF)) according to the present disclosure has an asymmetric structure, and is a non-polar hydrocarbon compound. Also, two asymmetric substituents of a 9-position fluorene group constitute a rigid cordo structure, such that the thermosetting resin prepared from the asymmetric bis(vinylbenzyl) fluorene according to the present disclosure has the characteristics of small dielectric dissipation factor, low dielectric constant, high glass transition temperature, and easy processing, and thus could be used as a resin material for high-frequency and high-speed substrates.

### BRIEF DESCRIPTION OF THE DRAWINGS

FIG. 1 shows a schematic diagram of the process of synthesizing an asymmetric bis(vinylbenzyl) fluorene through nucleophilic substitution reaction according to an embodiment of the present disclosure;

FIG. 2 shows the high-performance liquid chromatography (HPLC) chromatogram of the o,p-BVBF prepared in Example 1;

FIG. 3 shows the ¹H nuclear magnetic resonance (NMR) spectrum of the o,p-BVBF prepared in Example 1;

FIG. 4 shows the ¹³C NMR spectrum of the o,p-BVBF prepared in Example 1;

FIG. 5 shows the thermal analysis differential scanning calorimetry (DSC) diagram of the o,p-BVBF prepared in Example 1;

FIG. 6 shows the single crystal structure diagram of the o,p-BVBF prepared in Example 1; and

FIG. 7 shows the cell stacking diagram of the o,p-BVBF prepared in Example 1.

### DETAILED DESCRIPTION OF THE EMBODIMENTS

In the present disclosure, an asymmetric bis(vinylbenzyl) fluorene is synthesized, and the asymmetric bis(vinylbenzyl) fluorene is 9-(2-vinylbenzyl)-9′-(4-vinylbenzyl)-9H-fluorene (o,p-BVBF) having a structure shown in formula 1:

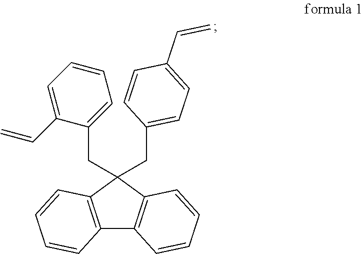 [SMILES: C=Cc1ccc(CC2(Cc3ccccc3C=C)c3ccccc3-c3ccccc32)cc1]

and
the 9-(2-vinylbenzyl)-9′-(4-vinylbenzyl)-9H-fluorene exhibits a thermal analysis melting endothermic peak in a temperature range of 169-175° C., and has a monoclinic crystal system and a space group of P2₁/c.

In the present disclosure, the term “asymmetric” in the “asymmetric bis(vinylbenzyl) fluorene” refers to the presence of different substituents at 9-position of a fluorene group.

In a specific embodiment of the present disclosure, the 9-(2-vinylbenzyl)-9′-(4-vinylbenzyl)-9H-fluorene exhibits the thermal analysis melting endothermic peak in the temperature range of 169-175° C., maybe 171-173° C.; the asymmetric bis(vinylbenzyl) fluorene may have a purity (HPLC) of greater than 98.5%.

The present disclosure provides two methods for preparing the 9-(2-vinylbenzyl)-9′-(4-vinylbenzyl)-9H-fluorene (o,p-BVBF).

The present disclosure provides a first method for preparing the asymmetric bis(vinylbenzyl) fluorene as described in above technical solutions, including:
mixing fluorene, a basic reagent, a polymerization inhibitor, and a polar aprotic solvent to obtain a fluorene-containing mixed system; and
dropwise adding 2-vinylbenzyl chloride and 4-vinylbenzyl chloride sequentially to the fluorene-containing mixed system, or dropwise adding a mixed solution of 2-vinylbenzyl chloride and 4-vinylbenzyl chloride to the fluorene-containing mixed system, and subjecting a resulting mixture to nucleophilic substitution reaction to obtain the asymmetric bis(vinylbenzyl) fluorene.

In a specific embodiment of the present disclosure, the basic reagent includes at least one selected from the group consisting of an alkali metal hydroxide, an alkali metal alkoxide, sodium hydride, and potassium hydride. In some embodiments, the basic reagent is specifically one selected from the group consisting of a mixture of the alkali metal hydroxide and the alkali metal alkoxide, the alkali metal hydroxide, the alkali metal alkoxide, the sodium hydride, and the potassium hydride. In some embodiments, the alkali metal hydroxide is sodium hydroxide or potassium hydroxide. In some embodiments, the alkali metal alkoxide is potassium tert-butoxide or sodium tert-butoxide. In some embodiments, a molar ratio of the fluorene to the basic reagent is in a range of 1:1.8 to 1:5, particularly 1:2 to 1:4.

In a specific embodiment of the present disclosure, the polymerization inhibitor includes at least one selected from the group consisting of nitromethane, nitrobenzene, o-nitrophenol, phenothiazine, 2-phenylnaphthylamine, hydroquinone, catechol, p-methoxyphenol, 2,6-di-tert-butyl-p-cresol, p-tert-butyl catechol, 2,2,6,6-tetramethylpiperidine oxynitride, and 2,2,6,6-tetramethyl-4-hydroxypiperidine oxynitride (701 polymerization inhibitor). In some embodiments, the polymerization inhibitor is specifically one selected from the group consisting of the nitromethane, the nitrobenzene, the o-nitrophenol, the phenothiazine, the 2-phenylnaphthylamine, the hydroquinone, the catechol, the p-methoxyphenol, the 2,6-di-tert-butyl-p-cresol, the p-tert-butyl catechol, the 2,2,6,6-tetramethylpiperidine oxynitride, and the 701 polymerization inhibitor. In some embodiments, the polymerization inhibitor is in an amount of 0.01-0.5%, particularly 0.1-0.4% of a mass of the vinyl benzyl chloride, the vinyl benzyl chloride being the 2-vinylbenzyl chloride and the 4-vinylbenzyl chloride.

In a specific embodiment of the present disclosure, the polar aprotic solvent includes one selected from the group consisting of dimethyl sulfoxide, sulfolane, N,N-dimethylformamide, N,N-diethylformamide, acetonitrile, or hexamethylphosphoramide. In the present disclosure, there is no special limitation on the amount of the polar aprotic solvent used, as long as raw materials could be mixed evenly.

In the present disclosure, there is no special requirement for the mixing of fluorene, the basic reagent, the polymerization inhibitor, and the polar aprotic solvent, as long as they could be mixed uniformly.

In the present disclosure, 2-vinylbenzyl chloride is firstly added dropwise to a fluorene-containing mixed system to generate a mono substituted 9-(2-vinylbenzyl) fluorene intermediate, and 4-vinylbenzyl chloride is then added dropwise for reaction, which results in a significantly increased yield of 9-(2-vinylbenzyl)-9′-(4-vinylbenzyl)-9H-fluorene (o,p-BVBF), compared with directly dropwise adding a mixed solution of the 2-vinylbenzyl chloride and the 4-vinylbenzyl chloride. If the 4-vinylbenzyl chloride is added dropwise first and then the 2-vinylbenzyl chloride is added for reaction, a main product would be 9,9′-bis(4-vinylbenzyl) fluorene (p,p-BVBF), while the yield of the 9-(2-vinylbenzyl)-9′-(4-vinylbenzyl)-9H-fluorene (o,p-BVBF) would be very low. It is because the steric hindrance of the 2-vinylbenzyl chloride is much greater than that of the 4-vinylbenzyl chloride during the reaction. The already presence of a 4-vinylbenzyl group at 9-position of a fluorenyl group makes it difficult to introduce 2-vinylbenzyl.

In a specific embodiment of the present disclosure, a molar ratio of the 2-vinylbenzyl chloride to the 4-vinylbenzyl chloride is in a range of 2-6:4-8, specifically 2:8, 3:7, 4:6, 5:5 or 6:4. In some embodiments, a ratio of a total amount in moles of the 2-vinylbenzyl chloride and the 4-vinylbenzyl chloride to an amount in moles of the fluorene is in a range of 1.8-2.2:1, specifically 2:1.

In a specific embodiment of the present disclosure, the nucleophilic substitution reaction is conducted at a temperature of 20-50° C., particularly 30-40° C. In some embodiments, the nucleophilic substitution reaction is conducted for 2-8 hours, particularly 3-7 hours. In a specific embodiment of the present disclosure, the nucleophilic substitution reaction is conducted under stirring. In the present disclosure, there is no special limitation on the stirring.

In a specific embodiment of the present disclosure, the method further includes: after the nucleophilic substitution reaction,
mixing a resulting system after the nucleophilic substitution reaction with ice water, and then conducting solid-liquid separation to obtain a solid;
washing the solid with water, then subjecting a washed solid to pulping, filtration, recrystallization, and drying sequentially to obtain the asymmetric bis(vinylbenzyl) fluorene.

In a specific embodiment of the present disclosure, a volume ratio of the resulting system after the nucleophilic substitution reaction to the ice water is in a range of 3:8-12, particularly 3:10-11. In some embodiments, the solid-liquid separation is performed by filtration. In the present disclosure, there is no special requirement for the number of the washing with the water, as long as a pH value of a filtrate after the washing with the water is neutral. In a specific embodiment of the present disclosure, a solvent used for pulping is an alcohol solvent. In some embodiments, the alcohol solvent includes one selected from the group consisting of methanol, ethanol, and isopropanol. In some embodiments, the pulping is conducted at a temperature of 0-40° C., particularly 10-30° C. In the present disclosure, there is no special limitation on the filtering, and conventional methods in this field may be adopted. In a specific embodiment of the present disclosure, a solvent used for the recrystallization includes one selected from the group consisting of toluene, ethylbenzene, xylene, and isopropylbenzene. In some embodiments, the drying is performed by vacuum drying. In some embodiments, the vacuum drying is performed at a temperature of 70-90° C., particularly 75-80° C. In the present disclosure, there is no special requirement for the time for the vacuum drying, as long as the solvent could be removed.

The present disclosure also provides a second method for preparing the asymmetric bis(vinylbenzyl) fluorene as described in above technical solutions, including:
mixing fluorene, a basic reagent, a polymerization inhibitor, a phase transfer catalyst, and a solvent to obtain a fluorene-containing phase transfer catalytic system; and
dropwise adding 2-vinylbenzyl chloride and 4-vinylbenzyl chloride sequentially to the fluorene-containing phase transfer catalytic system, or dropwise adding a mixed solution of 2-vinylbenzyl chloride and 4-vinylbenzyl chloride to the fluorene-containing phase transfer catalytic system, and subjecting a resulting mixture to nucleophilic substitution reaction under phase transfer catalysis conditions to obtain the asymmetric bis(vinylbenzyl) fluorene.

In a specific embodiment of the present disclosure, the basic reagent includes at least one selected from the group consisting of an alkali metal hydroxide and an alkali metal alkoxide. In some embodiments, the basic reagent is specifically one selected from the group consisting of a mixture of the alkali metal hydroxide and the alkali metal alkoxide, the alkali metal hydroxide, and the alkali metal alkoxide. In some embodiments, the alkali metal hydroxide is sodium hydroxide or potassium hydroxide. In some embodiments, the alkali metal alkoxide is potassium tert-butoxide or sodium tert-butoxide. In some embodiments, a molar ratio of the fluorene to the basic reagent is in a range of 1:1.8-5, particularly 1:2-4.

In a specific embodiment of the present disclosure, the polymerization inhibitor includes at least one selected from the group consisting of nitromethane, nitrobenzene, o-nitrophenol, phenothiazine, 2-phenylnaphthylamine, hydroquinone, catechol, p-methoxyphenol, 2,6-di-tert-butyl-p-cresol, p-tert-butyl catechol, 2,2,6,6-tetramethylpiperidine oxynitride, and 2,2,6,6-tetramethyl-4-hydroxypiperidine oxynitride (701 inhibitor). In some embodiments, the polymerization inhibitor is specifically one selected from the group consisting of nitromethane, nitrobenzene, o-nitrophenol, phenothiazine, 2-phenylnaphthylamine, hydroquinone, catechol, p-methoxyphenol, 2,6-di-tert-butyl-p-cresol, p-tert-butyl catechol, 2,2,6,6-tetramethylpiperidine oxynitride, and 701 inhibitor. In some embodiments, the polymerization inhibitor is in an amount of 0.01-0.5% particularly 0.1-0.4% of a mass of vinyl benzyl chloride, the vinyl benzyl chloride being the 2-vinyl benzyl chloride and the 4-vinyl benzyl chloride.

In a specific embodiment of the present disclosure, the phase transfer catalyst includes at least one selected from the group consisting of a quaternary ammonium salt, a quaternary phosphonium salt, and polyethylene glycol. In some embodiments, the phase transfer catalyst is specifically one selected from the group consisting of the quaternary ammonium salt, the quaternary phosphonium salt, and the polyethylene glycol. In some embodiments, the quaternary ammonium salt is one selected from the group consisting of tetrabutylammonium bromide (TBAB), tetrabutylammonium chloride (TBAC), benzyltriethylammonium bromide (BTEAB), benzyltriethylammonium chloride (BTEAC), hexadecyltriethylammonium bromide (HTEAB) and hexadecyltriethylammonium chloride (HTEAC). In some embodiments, the quaternary phosphonium salt is one selected from the group consisting of hexadecyltributylphosphonium bromide (HTBPB), hexadecyltributylphosphonium chloride (HTBPC), tetrabutylphosphonium bromide (TBPB), tetrabutylphosphonium chloride (TBPC), tetraphenylphosphonium bromide (TPPB) and tetraphenylphosphonium chloride (TPPC). In some embodiments, the polyethylene glycol is one selected from the group consisting of PEG-400, PEG-600, and PEG-800. In a specific embodiment of the present disclosure, a mass of the phase transfer catalyst is 5-25% particularly 10-20% of a mass of the fluorene.

In a specific embodiment of the present disclosure, the nucleophilic substitution reaction under the phase transfer catalysis conditions is nucleophilic substitution reaction under liquid-liquid phase transfer catalysis conditions or nucleophilic substitution reaction under solid-liquid phase transfer catalysis conditions.

In a specific embodiment of the present disclosure, under the condition that the nucleophilic substitution reaction under the phase transfer catalysis conditions is a reaction under the solid-liquid phase transfer catalysis conditions, a solvent used is an organic solvent, which includes one selected from the group consisting of dimethyl sulfoxide, sulfolane, N,N-dimethylformamide, N,N-diethylformamide, acetonitrile, and hexamethylphosphoramide.

In a specific embodiment of the present disclosure, under the condition that the nucleophilic substitution reaction under the phase transfer catalysis conditions is a reaction under the liquid-liquid phase transfer catalysis conditions, a solvent used is one selected from the group consisting of a mixture of an aromatic hydrocarbon and water and a mixture of an alkane and water. In some embodiments, the aromatic hydrocarbon is one selected from the group consisting of toluene, xylene, ethylbenzene, and isopropylbenzene. In some embodiments, the alkane is one selected from the group consisting of n-hexane, n-heptane, cyclohexane, methylcyclopentane, and petroleum ether.

In the present disclosure, 2-vinylbenzyl chloride is firstly added dropwise to generate a mono substituted 9-(2-vinylbenzyl) fluorene intermediate, and 4-vinylbenzyl chloride is then added dropwise for reaction, which results in a significantly increased yield of 9-(2-vinylbenzyl)-9′-(4-vinylbenzyl)-9H-fluorene (o,p-BVBF), compared with directly dropwise adding a mixed solution of the 2-vinylbenzyl chloride and the 4-vinylbenzyl chloride. If the 4-vinylbenzyl chloride is added dropwise first and then the 2-vinylbenzyl chloride is added for reaction, a main product would be 9,9′-bis(4-vinylbenzyl) fluorene (p,p-BVBF), while the yield of the 9-(2-vinylbenzyl)-9′-(4-vinylbenzyl)-9H-fluorene (o,p-BVBF) would be very low. It is because the steric hindrance of the 2-vinylbenzyl chloride is much greater than that of the 4-vinylbenzyl chloride during the reaction. The already presence of a 4-vinylbenzyl group at 9-position of a fluorenyl group makes it difficult to introduce 2-vinylbenzyl. Therefore, the 2-vinylbenzyl chloride is added dropwise first, and after reaction for a period of time, the 4-vinylbenzyl chloride is then added dropwise for reaction, which is beneficial for improving the yield of the 9-(2-vinylbenzyl)-9′-(4-vinylbenzyl)-9H-fluorene (o,p-BVBF).

In a specific embodiment of the present disclosure, a molar ratio of the 2-vinylbenzyl chloride to the 4-vinylbenzyl chloride is in a range of 2-6:4-8, specifically 2:8, 3:7, 4:6, 5:5, or 6:4. In some embodiments, a ratio of a total amount in moles of the 2-vinylbenzyl chloride and the 4-vinylbenzyl chloride to an amount in moles of the fluorene is in a range of 1.8-2.2:1, specifically 2:1.

In a specific embodiment of the present disclosure, the nucleophilic substitution reaction under the phase transfer catalysis conditions is performed at a temperature of 25-75° C., particularly 30-70° C. In some embodiments, the nucleophilic substitution reaction under the phase transfer catalysis conditions is performed for 8 hours to 18 hours, particularly 10 hours to 15 hours. In a specific embodiment of the present disclosure, the nucleophilic substitution reaction under the phase transfer catalysis conditions is conducted under good stirring conditions.

In a specific embodiment of the present disclosure, the method further includes: after the nucleophilic substitution reaction under the phase transfer catalysis conditions,
distilling a resulting system after the nucleophilic substitution reaction under the phase transfer catalysis conditions to remove the solvent therein, then adding water and toluene thereto, and conducting extraction to obtain an organic phase;
subjecting the organic phase to concentration, washing, pulping, filtration, recrystallization, and drying sequentially to obtain the asymmetric bis(vinylbenzyl) fluorene.

In a specific embodiment of the present disclosure, a volume ratio of the water to the toluene for the extraction is in a range of 1:0.8-1.2, particularly is 1:1. In some embodiments, the concentration is performed by vacuum distillation. In the present disclosure, there is no special requirement for the vacuum distillation, as long as the solvent could be removed. In a specific embodiment of the present disclosure, a solvent used for the washing is a saturated ammonium chloride solution. In the present disclosure, there is no special requirement for the number of the washing, as long as a pH value of a filtrate after the washing is neutral. In a specific embodiment of the present disclosure, a solvent used for the pulping is an alcohol solvent having not more than five carbon atoms. In some embodiments, the alcohol solvent having not more than five carbon atoms includes one selected from the group consisting of methanol, ethanol, and isopropanol. In some embodiments, the pulping is performed at a temperature of 0-40° C., particularly 10-30° C. In the present disclosure, there is no special limitation on the filtration, and conventional means in this field may be adopted. In a specific embodiment of the present disclosure, a solvent used for the recrystallization includes one selected from the group consisting of toluene, ethylbenzene, xylene, and isopropylbenzene. In some embodiments, the drying is performed by vacuum drying. In some embodiments, the vacuum drying is performed at a temperature of 70-90° C., particularly 75-80° C. In the present disclosure, there is no special requirement for the time of the vacuum drying, as long as the solvent could be removed.

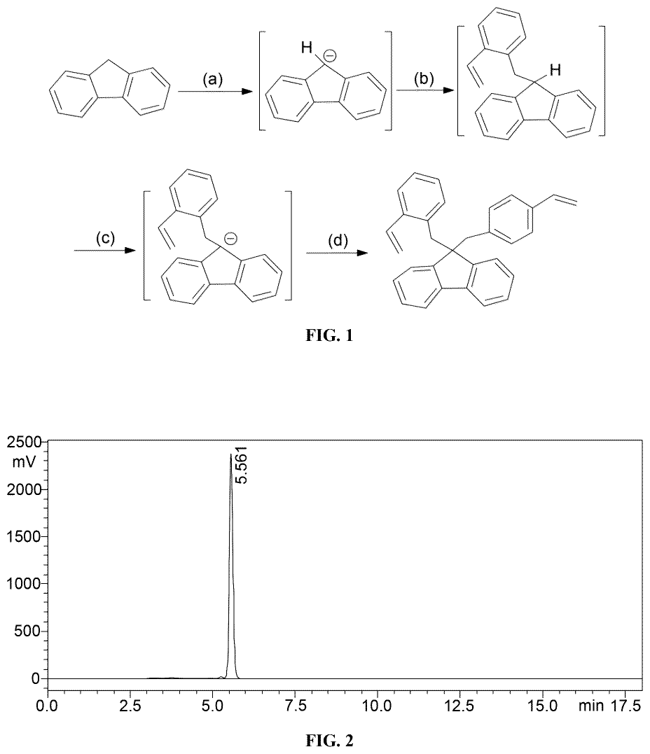

FIG. 1 shows a schematic diagram of the process of synthesizing an asymmetric bis(vinylbenzyl) fluorene through nucleophilic substitution reaction according to an embodiment of the present disclosure. In FIG. 1, (a) represents adding a basic agent; (b) represents adding 2-vinylbenzyl chloride; (c) represents adding a basic agent; and (d) represents adding 4-vinylbenzyl chloride.

The present disclosure also provides a thermosetting resin, including at least one selected from the group consisting of a thermosetting hydrocarbon resin and a thermosetting cross-linked resin, where the thermosetting hydrocarbon resin includes at least one selected from the group consisting of a thermosetting hydrocarbon resin prepared by using 9-(2-vinylbenzyl)-9′-(4-vinylbenzyl)-9H-fluorene as a raw material and a thermosetting hydrocarbon resin prepared by using 9-(2-vinylbenzyl)-9′-(4-vinylbenzyl)-9H-fluorene and 9,9′-bis(4-vinylbenzyl)-9H-fluorene as raw materials;
the thermosetting cross-linked resin includes a thermosetting cross-linked resin prepared by using a cross-linking agent and an alkenyl-terminated polyphenylene ether as raw materials, the cross-linking agent includes at least one selected from the group consisting of 9-(2-vinylbenzyl)-9′-(4-vinylbenzyl)-9H-fluorene and 9,9′-bis(4-vinylbenzyl)-9H-fluorene, and the alkenyl-terminated polyphenylene ether has a structure selected from the group consisting of formula 2, formula 3, formula 4, and formula 5,

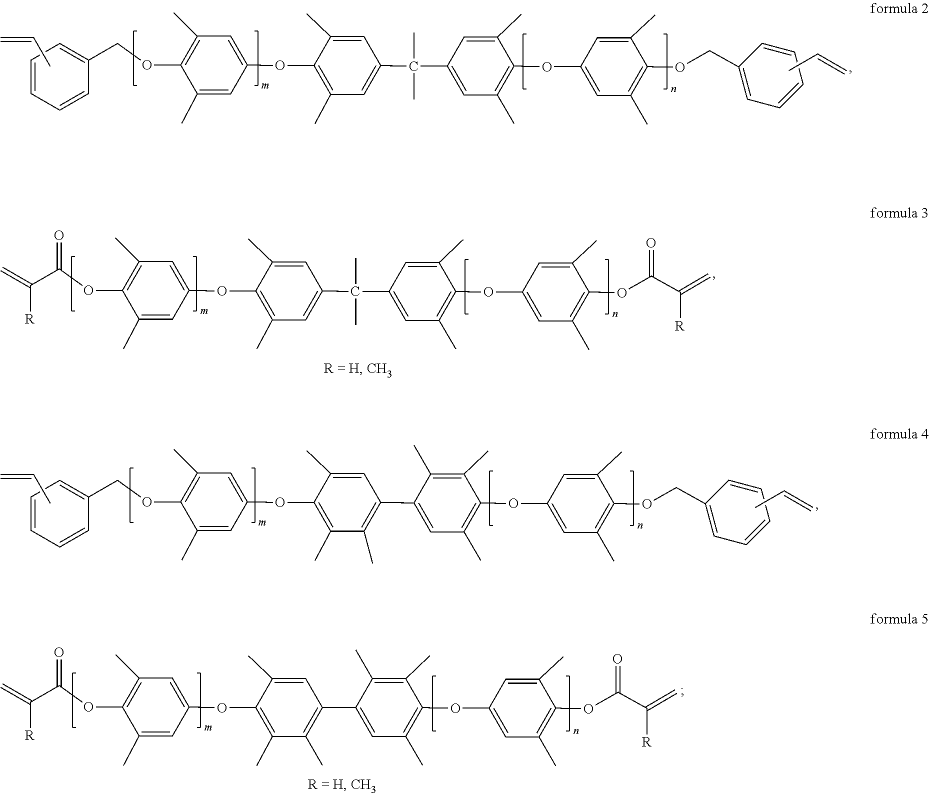

and
the 9-(2-vinylbenzyl)-9′-(4-vinylbenzyl)-9H-fluorene is the asymmetric bis(vinylbenzyl) fluorene as described in above technical solutions, or the asymmetric bis(vinylbenzyl) fluorene prepared by the method as described in above technical solutions.

In a specific embodiment of the present disclosure, the thermosetting hydrocarbon resin prepared by using the 9-(2-vinylbenzyl)-9′-(4-vinylbenzyl)-9H-fluorene as the raw material has a low dielectric constant (Dk (10GH) of 2.8), a low dielectric dissipation factor (Df (10GH) of 0.00030), and a high glass transition temperature (Tg of 360° C.).

In a specific embodiment of the present disclosure, a molar percentage of o,p-BVF in the raw materials of the thermosetting hydrocarbon resin prepared by using the 9-(2-vinylbenzyl)-9′-(4-vinylbenzyl)-9H-fluorene (o,p-BVF) and the 9,9′-bis(4-vinylbenzyl)-9H-fluorene (p,p-BVBF) as the raw materials ranges from 10% to 60%, particularly from 25% to 50%. In the present disclosure, the 9,9′-bis(4-vinylbenzyl)-9H-fluorene is a symmetric bis(vinylbenzyl) fluorene, where symmetric refers to the presence of same substituents at 9-position of a fluorene group. A structural formula of the 9,9′-bis(4-vinylbenzyl)-9H-fluorene is

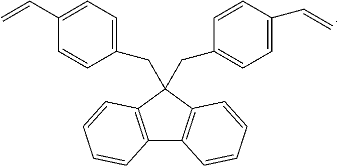 [SMILES: C=Cc1ccc(CC2(Cc3ccc(C=C)cc3)c3ccccc3-c3ccccc32)cc1]

In a specific embodiment of the present disclosure, the thermosetting hydrocarbon resin prepared by using the 9-(2-vinylbenzyl)-9′-(4-vinylbenzyl)-9H-fluorene and the 9,9′-bis(4-vinylbenzyl)-9H-fluorene as the raw materials exhibits low dielectric constant, low dielectric dissipation factor, and relatively high glass transition temperature. For example, under the condition that a molar ratio of the o,p-BVF to the p,p-BVBF is 1:1, the resulting thermosetting hydrocarbon resin exhibits a dielectric constant Dk (10GH) of 2.8, a dielectric dissipation factor Df (10GH) of 0.00042, and a glass transition temperature of 352° C.

In a specific embodiment of the present disclosure, in the raw materials of the thermosetting cross-linked resin prepared by using the cross-linking agent and the alkenyl-terminated polyphenylene ether as the raw materials, the alkenyl-terminated polyphenylene ether has a number average molecular weight (Mn) of 1800-2300. In some embodiments, the alkenyl-terminated polyphenylene ether is specifically vinylbenzyl-terminated modified polyphenylene ether, and the vinylbenzyl-terminated modified polyphenylene ether has a structural formula of:

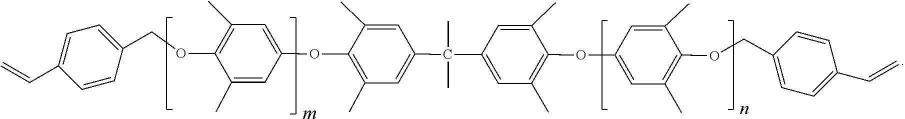

In a specific embodiment of the present disclosure, the vinylbenzyl-terminated modified polyphenylene ether is prepared by a process including:
subjecting 2,6-dimethylphenol, a diphenol, and a catalyst to oxidative coupling copolymerization under oxygen conditions to obtain hydroxy polyphenylene ether; and
subjecting the hydroxyl polyphenylene ether and vinyl benzyl chloride to reaction under phase transfer conditions, and then adding methanol to precipitate a solid to obtain the vinylbenzyl-terminated modified polyphenylene ether.

In a specific embodiment of the present disclosure, the catalyst includes a copper amine complex catalyst. In some embodiments, the copper amine complex catalyst includes one selected from the group consisting of N,N′-tetra-tert-butyl ethylenediamine, N-methylbutylamine, cuprous bromide, cuprous chloride, copper chloride, and copper bromide. In some embodiments, the hydroxy polyphenylene ether has a number average molecular weight of 1600-2000.

In a specific embodiment of the present disclosure, under the condition that the 9-(2-vinylbenzyl)-9′-(4-vinylbenzyl)-9H-fluorene and the 9,9′-bis(4-vinylbenzyl)-9H-fluorene are used as cross-linking agents to prepare the thermosetting cross-linked resin, a mass percentage content of the cross-linking agents in the raw materials ranges from 10% to 50%, particularly from 20% to 40%. In some embodiments, a molar percentage of the 9-(2-vinylbenzyl)-9′-(4-vinylbenzyl)-9H-fluorene (o,p-BVF) in the cross-linking agents ranges from 10% to 60%, particularly from 25% to 50%.

The thermosetting resin according to the present disclosure has a low dielectric constant, a low dielectric dissipation factor, and an improved glass transition temperature, thus having great application prospects as a resin material for high-frequency substrates.

The present disclosure provides a method for preparing the thermosetting resin as described in above technical solutions, including:
dissolving the raw material(s) and an initiator in toluene to obtain a mixed solution; and
removing the toluene from the mixed solution, and then conducting melting thermal curing to obtain the thermosetting resin.

In a specific embodiment of the present disclosure, the initiator includes a peroxide, the peroxide includes di-tert-butyl peroxide, tert-butyl hydroperoxide, isopropylbenzene peroxide, benzoyltert-butyl peroxide, di(tert-butyl peroxyisopropyl)benzene, benzoyl peroxide, di(4-methylbenzoyl) peroxide, dilauroyl peroxide, 1,1-di(tert-butyl peroxide) cyclohexane, 1,1-di(tert-butyl peroxide)-3,3,5-trimethylcyclohexane, 2,5-dimethyl-2,5-di(tert-butylperoxide) hexane, 3,3,5,7,7-pentamethyl-1,2,4-trioxane (T311), tert-butyl peroxy-2-ethylhexanoate, tert-butyl peroxy-2-ethylhexyl carbonate ester. In some embodiments, a mass ratio of the raw materials to the initiator is in a range of 1:0.001-0.008, particularly 1:0.003-0.005.

In a specific embodiment of the present disclosure, means for removing the toluene from the mixed solution is vacuum distillation.

In a specific embodiment of the present disclosure, the melting thermal curing is performed at a temperature of 200-240° C., particularly 210-230° C. In some embodiments, the melting thermal curing is conducted at a pressure of 70-80 mmHg, particularly 75-78 mmHg. In some embodiments, the melting thermal curing is conducted for 80-120 minutes, particularly 90-110 minutes.

The present disclosure provides use of the thermosetting resin as described in above technical solutions or the thermosetting resin prepared by the method as described in above technical solutions as a resin material for a high-frequency and high-speed substrate.

In order to further illustrate the present disclosure, the technical solutions according to the present disclosure will be described in detail below in conjunction with examples, but they should not be construed as limiting the scope of the present disclosure.

Instruments and methods used for analyses and tests in the Examples:
1. Purity determination method (HPLC): U.S. Agillent 1260 high-pressure liquid chromatograph; column type: Kromasil 100-5 C18 250 cm×4.6 mm; a mobile phase being acetonitrile/methanol in a volume ratio of 9:1; a flow rate being 0.8 mL/min; a detection wavelength being 254 nm; a sample injection volume being 2 L; a pump mode being binary high-pressure gradient.
2. Thermal analysis method: Pyris1 Thermal Analyzer (from Perkin Elemer company) was used for DSC measurement according to JY/T0589.3-2020 rules, with a heating temperature range of 50-200° C. and a heat-up rate of 10° C./min.
3. Nuclear magnetic resonance spectrometry: Bruker AV 400 nuclear magnetic resonance spectrometer, with DMSO-d₆ as a solvent, and TMS as an internal standard.
4. Determination of single-crystal structure: Bruker D8 Venture single-crystal diffractometer, JY/T0588-2020 General Rules for Analysis Method of Molecular Structure.
5. Determination of dielectric constant Dk (10 GHz) and dielectric dissipation factor Df (10 GHz): A molten sample pouring method (conditions of thermal solidification: a temperature being 220° C., a pressure being 70-80 mmHg, and a time being 100 min) was used to prepare 80 mm×80 mm×0.4 mm resin sheets, and measurement was performed by using U.S. Agilent N5230A vector network analyzer at a frequency of 10 GHz (SPDR).
6. Determination of polymer molecular weight: U.S. Agillent 1260 gel chromatographic instrument, tetrahydrofuran as a mobile phase, polystyrene as a standard.
7. Determination of glass transition temperature of polymer: determined by using Perkin Elemer Differential Scanning Calorimeter DSC 4000 instrument.

The 2-vinylbenzyl chloride (with a HPLC purity of 99.0%), 4-vinylbenzyl chloride (with a HPLC purity of 99.5%), and 1,2-bis(4-vinylphenyl)ethane (BVPE) (with a HPLC purity of 99.5%) used in examples were all produced by Shandong Xingshun New Material Co., Ltd., China.

In Examples 1-7, 9-(2-vinylbenzyl)-9′-(4-vinylbenzyl)-9H-fluorene (o,p-BVBF) was prepared, and in Examples 8-13, thermosetting resins were prepared.

### Example 1

300 mL of anhydrous dimethyl sulfoxide, 1.10 mol of potassium tert-butoxide, and 0.3 g of 701 polymerization inhibitor were added to a reaction flask. A resulting mixture was stirred under a nitrogen flow for 30 minutes, followed by adding 0.5 mol of fluorene. After complete dissolution through stirring, 0.4 mol of 2-vinylbenzyl chloride was added dropwise thereto. A resulting mixture was subjected to reaction at a temperature of 30-35° C. under stirring at 350 rpm for 1.0 hour. Subsequently, 0.6 mol of 4-vinylbenzyl chloride was added dropwise under continuous stirring, and the reaction was continued for 2.5 hours. A resulting reaction system was slowly poured into 1000 mL of ice water, and a solid precipitated. The precipitated solid was collected by filtration, washed with water until a resulting filtrate was neutral, and subjected to pulping (30° C.) with 500 mL of methanol twice. A resulting system was filtered to obtain a solid, and the solid was subjected to recrystallization twice with toluene. A resulting solid was then vacuum-dried to a constant weight at 80° C., obtaining 144.5 g of 9-(2-vinylbenzyl)-9′-(4-vinylbenzyl)-9H-fluorene (o,p-BVBF), with a yield of 72.5%.

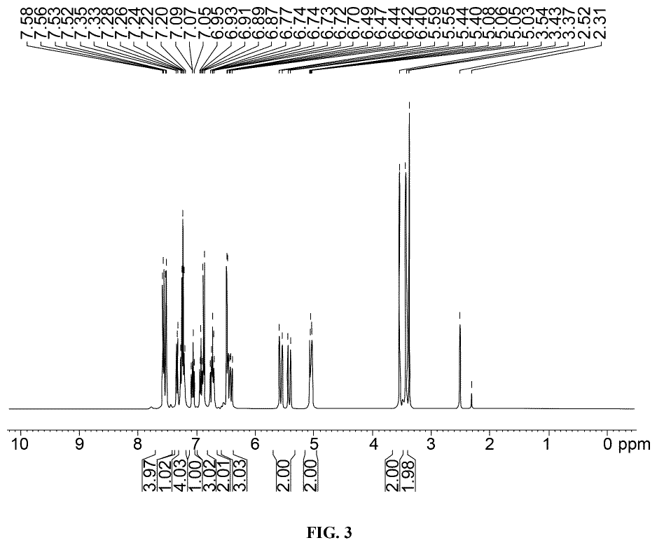

The obtained o,p-BVBF appeared as a white crystal, and was subjected to high-performance liquid chromatography (HPLC) analysis. An obtained high-performance liquid chromatogram is shown in FIG. 2. Table 1 shows the HPLC peak information. A HPLC purity of the o,p-BVBF was determined to be 99.15% based on HPLC analysis results.

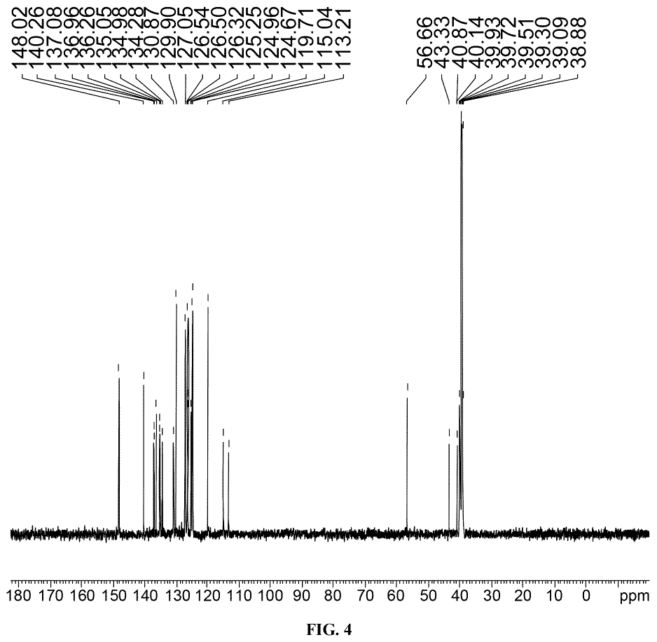

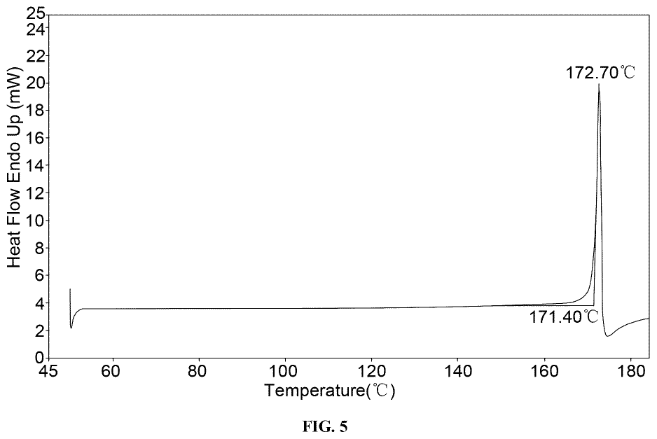

Nuclear magnetic resonance (NMR) characterization of o,p-BVBF was conducted. Both ¹H and ¹³C NMR spectra were obtained, as shown in FIGS. 3 and 4. FIG. 3 shows the ¹H NMR spectrum of the o,p-BVBF and FIG. 4 shows the ¹³C NMR spectrum of the o,p-BVBF.

¹H NMR (400 MHz, DMSO-d₆) δ: 3.43 (s, 2H, CH₂), 3.54 (s, 2H, CH₂), 5.03-5.08 (m, 2H, 2× alkene bond hydrogen), 5.40-5.59 (m, 2H, 2× alkene bond hydrogen), 6.40-6.42 (m, 1H, 1× alkene bond hydrogen), 6.49 (d, 2H, 2× benzene ring hydrogen), 6.70-6.77 (m, 2H, 2× benzene ring hydrogen), 6.97-7.01 (m, 3H, 2× benzene ring hydrogen, 1× alkene bond hydrogen), 6.87-6.95 (m, 1H, 1× benzene ring hydrogen), 7.05-7.20 (m, 4H, 4× fluorene ring hydrogen), 7.22-7.25 (m, 1H, 1× benzene ring hydrogen), 7.33-7.58 (m, 4H, 4× fluorene ring hydrogen).

¹³C NMR (100 MHz, DMSO-d₆) δ: 40.9 (CH₂), 43.3 (CH₂), 56.7 (9-fluorene C), 113.2, 115.0, 119.7, 124.7, 124.9, 125.3, 126.3, 126.5, 126.7, 127.1, 129.9, 130.9, 134.3, 134.9, 135.1, 136.3, 136.9, 137.0, 140.3, 148.0 (benzene ring C, fluorene ring C, vinyl C).

The ¹H NMR spectrum and ¹³C NMR spectrum completely confirmed the structure of the o,p-BVBF.

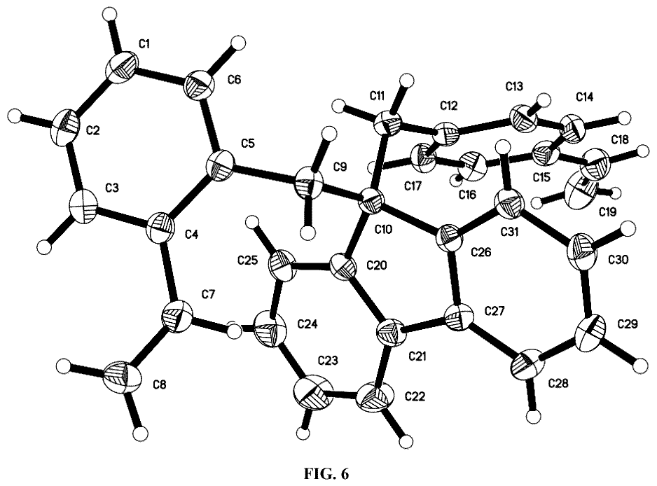

The prepared o,p-BVBF was tested using a Pyris1 thermal analyzer. An obtained DSC spectrum is shown in FIG. 5. It can be seen from FIG. 5 that a melting point of the o,p-BVBF is 171.4-172.7° C.

FIG. 6 shows the single crystal structure diagram of the o,p-BVBF, where

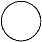

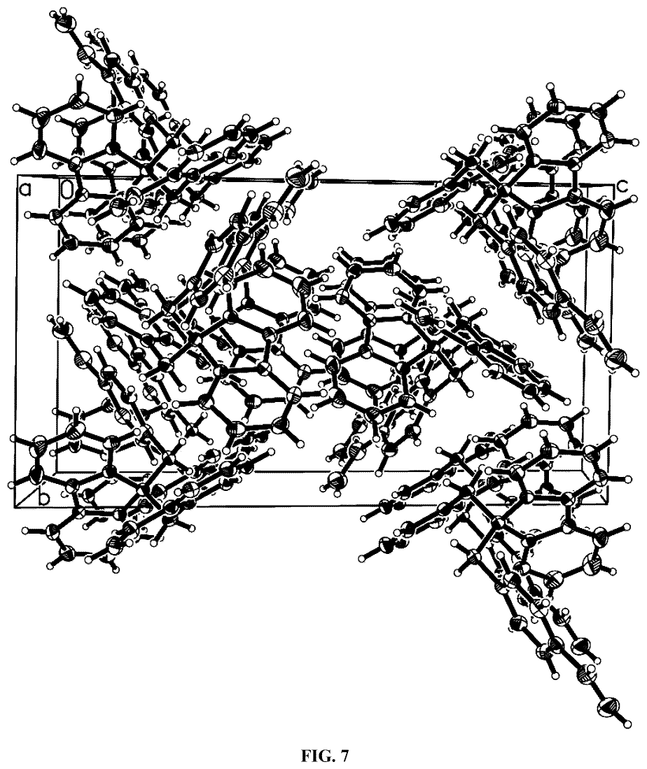

represents H atoms; FIG. 7 shows the cell stacking diagram of the o,p-BVBF. Crystallographic parameters of the o,p-BVBF are shown in Table 2, and bond length and bond angle data of the o,p-BVBF are shown in Table 3; determined results of single crystal structure further confirm the molecular structure of the o,p-BVBF.

TABLE 1
HPLC peak information of o,p-BVBF

| | Retention | | | Peak | |
|---|---|---|---|---|---|
| Peaks | time/minute | Type | Peak area | height/mV | Peak area/% |
| 1 | 4.135 | VB | 0.2405 | 308 | 0.011 |
| 2 | 4.370 | VB | 0.9071 | 1311 | 0.050 |
| 3 | 4.526 | BV | 0.3897 | 555 | 0.021 |
| 4 | 5.262 | VV | 10.5175 | 13630 | 0.577 |
| 5 | 5.561 | VB | 1808.5450 | 2374847 | 99.150 |
| 6 | 6.509 | VB | 0.1061 | 131 | 0.006 |
| 7 | 7.548 | BB | 0.8422 | 708 | 0.046 |

TABLE 2
Crystallographic parameters of o,p-BVBF

| Compound | o,p-BVBF | |
|---|---|---|
| Molecular formula | C₃₁H₂₆ | |
| Molecular weight | 398.52 | |
| Test temperature | 223.00 | K |
| Wavelength | 1.54178 | Å |
| Crystal system | Monoclinic crystal system | |
| Space group | P2₁/c | |
| Cell parameters | a = 10.7721(4) Å □ = 90° | |
| | b = 10.7089(4) Å | |
| | □ = 101.173(2)°. | |
| | c = 19.7979(8) Å □ = 90° | |
| Cell volume | 2240.55(15) | Å³ |
| Z | 4 | |
| Density | 1.181 megagram/cubic meter | |
| Absorption | 0.501 | mm⁻¹ |
| coefficient | | |
| F(000) | 848.0 | |
| Crystal size | 0.15 × 0.13 × 0.10 | mm³ |

| Θ angle range | 4.183-68.278° |
|---|---|
| Index range | −11 ≤ h ≤ 12, −12 ≤ k ≤ |
| | 11, −22 ≤ l ≤ 23 |
| Number of | 18475 |
| collected diffraction | |
| points | |
| Number of | 4058[R(int) = 0.0467] |
| independent | |
| diffraction points | |
| Θ = 68.278° integrity | 99.2% |
| Absorption | Semi-empirical from |
| correction | equivalents |
| Maximum and | 0.753 and 0.639 |
| minimum | |
| transmittance | |
| Refining method | Full-matrix least-squares on |
| | F² |
| Number of data | 4058/0/280 |
| points/number of | |
| limitations | |
| added/number of | |
| refined parameters | |
| GooF value | 1.061 |
| Deviation factor | R₁ = 0.0401, wR₂ = 0.1054 |
| [I > 2sigma(I)] | |
| Deviation factor | R₁ = 0.0547, wR₂ = 0.1128 |
| (all data) | |
| Residual electron | 0.15 and −0.19 e · Å⁻³ |
| cloud density | |

Table 3 Bond length [Å] and bond angle [°] of o,p-BVBF

| | | | |
|---|---|---|---|
| C(1)-C(6) | 1.387(2) | C(6)-C(5)-C(9) | 119.26(13) |
| C(2)-C(3) | 1.372(2) | C(1)-C(6)-C(5) | 121.67(14) |
| C(3)-C(4) | 1.3994(19) | C(8)-C(7)-C(4) | 127.53(15) |
| C(4)-C(5) | 1.4061(19) | C(5)-C(9)-C(10) | 116.23(10) |
| C(4)-C(7) | 1.478(2) | C(11)-C(10)-C(9) | 110.07(10) |
| C(5)-C(6) | 1.3906(19) | C(20)-C(10)-C(9) | 112.41(10) |
| C(5)-C(9) | 1.5124(17) | C(20)-C(10)-C(11) | 112.22(10) |
| C(7)-C(8) | 1.306(2) | C(26)-C(10)-C(9) | 108.04(10) |
| C(9)-C(10) | 1.5679(17) | C(26)-C(10)-C(11) | 112.23(10) |
| C(10)-C(11) | 1.5473(17) | C(26)-C(10)-C(20) | 101.57(10) |
| C(10)-C(20) | 1.5203(17) | C(12)-C(11)-C(10) | 114.30(10) |
| C(10)-C(26) | 1.5203(16) | C(13)-C(12)-C(11) | 120.89(12) |
| C(11)-C(12) | 1.5085(18) | C(13)-C(12)-C(17) | 117.43(13) |
| C(12)-C(13) | 1.3880(18) | C(17)-C(12)-C(11) | 121.68(12) |
| C(12)-C(17) | 1.390(2) | C(14)-C(13)-C(12) | 121.35(14) |
| C(13)-C(14) | 1.384(2) | C(13)-C(14)-C(15) | 121.19(13) |
| C(14)-C(15) | 1.385(2) | C(14)-C(15)-C(16) | 117.77(14) |
| C(15)-C(16) | 1.396(2) | C(14)-C(15)-C(18) | 119.89(14) |
| C(15)-C(18) | 1.471(2) | C(16)-C(15)-C(18) | 122.34(15) |
| C(16)-C(17) | 1.384(2) | C(17)-C(16)-C(15) | 120.77(14) |
| C(18)-C(19) | 1.284(3) | C(16)-C(17)-C(12) | 121.48(13) |
| C(20)-C(21) | 1.3927(19) | C(19)-C(18)-C(15) | 127.95(19) |
| C(20)-C(25) | 1.3886(18) | C(21)-C(20)-C(10) | 110.43(11) |
| C(21)-C(22) | 1.386(2) | C(25)-C(20)-C(10) | 129.29(12) |
| C(21)-C(27) | 1.4690(18) | C(25)-C(20)-C(21) | 120.27(12) |
| C(22)-C(23) | 1.387(3) | C(20)-C(21)-C(27) | 108.87(11) |
| C(23)-C(24) | 1.381(3) | C(22)-C(21)-C(20) | 120.55(13) |
| C(24)-C(25) | 1.383(2) | C(22)-C(21)-C(27) | 130.58(14) |
| C(26)-C(27) | 1.3945(17) | C(21)-C(22)-C(23) | 118.61(16) |
| C(26)-C(31) | 1.3818(18) | C(24)-C(23)-C(22) | 120.92(15) |
| C(27)-C(28) | 1.3870(19) | C(23)-C(24)-C(25) | 120.61(15) |
| C(28)-C(29) | 1.379(2) | C(24)-C(25)-C(20) | 118.95(14) |
| C(29)-C(30) | 1.384(2) | C(27)-C(26)-C(10) | 110.77(10) |
| C(30)-C(31) | 1.3887(19) | C(31)-C(26)-C(10) | 128.93(11) |
| | | C(31)-C(26)-C(27) | 120.29(12) |
| C(2)-C(1)-C(6) | 119.74(14) | C(26)-C(27)-C(21) | 108.23(11) |
| C(3)-C(2)-C(1) | 119.58(13) | C(28)-C(27)-C(21) | 131.19(12) |
| C(2)-C(3)-C(4) | 121.93(14) | C(28)-C(27)-C(26) | 120.56(12) |
| C(3)-C(4)-C(5) | 118.60(13) | C(29)-C(28)-C(27) | 118.83(13) |
| C(3)-C(4)-C(7) | 119.52(13) | C(28)-C(29)-C(30) | 120.79(13) |
| C(5)-C(4)-C(7) | 121.88(12) | C(29)-C(30)-C(31) | 120.63(13) |
| C(4)-C(5)-C(9) | 122.26(12) | C(26)-C(31)-C(30) | 118.88(13) |

### Example 2

300 mL of anhydrous dimethyl sulfoxide, 1.10 mol of potassium tert-butoxide, and 0.3 g of polymerization inhibitor 701 were added to a reaction flask. A resulting mixture was stirred under a nitrogen flow for 30 minutes, followed by adding 0.5 mol of fluorene. After complete dissolution through stirring, 0.3 mol of 2-vinylbenzyl chloride was added dropwise thereto. A resulting mixture was subjected to reaction at 35° C. under stirring at 350 rpm for 1.0 hour. Subsequently, 0.7 mol of 4-vinylbenzyl chloride was added dropwise under stirring, and the reaction was continued under stirring for 2.5 hours. A resulting reaction system was slowly poured into 1000 mL of ice water, and a solid precipitated. The precipitated solid was collected by filtration, washed with water until a resulting filtrate was neutral, and subjected to pulping (30° C.) with 500 mL of methanol twice. Aresulting system was filtered to obtain a solid, and the solid was subjected to recrystallization twice with toluene. A resulting solid was then vacuum-dried at 80° C. to a constant weight, obtaining 110.8 g of 9-(2-vinylbenzyl)-9′-(4-vinylbenzyl)-9H-fluorene (o,p-BVBF) as a white crystal, having a purity (HPLC) of 99.1%, with a yield of 55.6%.

### Example 3

300 mL of anhydrous dimethyl sulfoxide, 1.10 mol of potassium tert-butoxide, and 0.3 g of polymerization inhibitor 701 were added to a reaction flask. A resulting mixture was stirred under a nitrogen flow for 30 minutes, followed by adding 0.5 mol of fluorene. After complete dissolution through stirring, 0.5 mol of 2-vinylbenzyl chloride was added dropwise thereto. A resulting mixture was subjected to reaction at 35° C. under stirring at 350 rpm for 1.0 hour. Subsequently, 0.5 mol of 4-vinylbenzyl chloride was added dropwise under stirring, and the reaction was continued under stirring for 2.5 hours. A resulting reaction system was slowly poured into 1000 mL of ice water, and a solid precipitated. The precipitated solid was collected by filtration, washed with water until a resulting filtrate was neutral, and subjected to pulping (30° C.) with 500 mL of methanol twice. Aresulting system was filtered to obtain a solid, and the solid was subjected to recrystallization twice with toluene. A resulting solid was then vacuum-dried at 80° C. to a constant weight, obtaining 126.7 g of 9-(2-vinylbenzyl)-9′-(4-vinylbenzyl)-9H-fluorene (o,p-BVBF) as a white crystal, having a purity (HPLC) of 99.2%, with a yield of 63.6%.

### Example 4

300 mL of anhydrous dimethyl sulfoxide, 1.10 mol of potassium tert-butoxide, and 0.3 g of polymerization inhibitor 701 were added to a reaction flask. A resulting mixture was stirred under a nitrogen flow for 30 minutes, followed by adding 0.5 mol of fluorene. After complete dissolution through stirring, a mixed solution of 0.4 mol of 2-vinylbenzyl chloride and 0.6 mol of 4-vinylbenzyl chloride was added dropwise thereto. A resulting mixture was subjected to reaction at 35° C. under stirring at 350 rpm for 3.5 hours. A resulting reaction system was slowly poured into 1000 mL of ice water, and a solid precipitated. The precipitated solid was collected by filtration, washed with water until a resulting filtrate was neutral, and subjected to pulping (30° C.) with 500 mL of methanol twice. A resulting system was filtered to obtain a solid, and the solid was subjected to recrystallization twice with toluene. A resulting solid was then vacuum-dried at 80° C. to a constant weight, obtaining 90.6 g of 9-(2-vinylbenzyl)-9′-(4-vinylbenzyl)-9H-fluorene (o,p-BVBF) as a white crystal, having a purity (HPLC) of 98.5%, with a yield of 45.5%.

### Example 5

500 mL of toluene and 0.5 mol of fluorene were added to a reaction flask, and stirred until dissolution. 180 g of a pre-prepared 50% aqueous sodium hydroxide solution, 8.5 g of tetrabutylammonium bromide (TBAB), and 0.3 g of polymerization inhibitor 701 were added under stirring thereto. At 40° C., 0.4 mol of 2-vinylbenzyl chloride was added dropwise thereto under stirring and a nitrogen flow, and a resulting mixture was subjected to reaction under stirring at 350 rpm for 4.0 hours. 0.6 mol of 4-vinylbenzyl chloride was then added dropwise thereto under stirring, and the reaction was continued under stirring for 10 hours. After cooling to room temperature, 500 mL of water was slowly added thereto under stirring, and an organic phase was separated, washed twice with water (500 mL each time), then washed with 500 mL of saturated aqueous ammonium chloride solution, and finally washed with water until neutral. A resulting organic phase was separated, toluene therein was removed under reduced pressure, and a residue was subjected to pulping (30° C.) with 500 mL of methanol twice. Aresulting system was filtered to obtain a solid, and the solid was subjected to recrystallization twice with toluene. A resulting solid was then vacuum-dried at 80° C. to a constant weight, obtaining 124.3 g of 9-(2-vinylbenzyl)-9′-(4-vinylbenzyl)-9H-fluorene (o,p-BVBF) as a white crystal, having a purity (HPLC) of 98.5%, with a yield of 62.4%.

### Example 6

500 mL of toluene and 0.5 mol of fluorene were added to a reaction flask, and stirred until dissolution. 180 g of a pre-prepared 50% aqueous sodium hydroxide solution, 8.5 g of tetrabutylammonium bromide (TBAB), and 0.3 g of polymerization inhibitor 701 were added under stirring thereto. At 40° C., a mixed solution of 0.5 mol of 2-vinylbenzyl chloride and 0.5 mol of 4-vinylbenzyl chloride was added dropwise thereto under stirring and a nitrogen flow, and a resulting mixture was subjected to reaction under stirring at 350 rpm for 14.0 hours. After cooling to room temperature, 500 mL of water was slowly added thereto under stirring, and an organic phase was separated, washed twice with water (500 mL each time), then washed with 500 mL of saturated ammonium chloride solution, and finally washed with water until neutral. A resulting organic phase was separated, toluene therein was removed under reduced pressure, and a residue was subjected to pulping (30° C.) with 500 mL of methanol twice. Aresulting system was filtered to obtain a solid, and the solid was subjected to recrystallization twice with toluene. A resulting solid was then vacuum-dried at 80° C. to a constant weight, obtaining 84.7 g of 9-(2-vinylbenzyl)-9′-(4-vinylbenzyl)-9H-fluorene (o,p-BVBF) as a white crystal, having a purity (HPLC) of 98.5%, with a yield of 42.5%.

### Example 7

500 mL of acetonitrile, 1.2 mol of a potassium hydroxide powder, 30 g of PEG-400, and 0.3 g of polymerization inhibitor 701 were added to a reaction flask, and 0.5 mol (83 g) of fluorene was added thereto under stirring. At 40° C., 0.4 mol of 2-vinylbenzyl chloride was added dropwise thereto under stirring and a nitrogen flow, and a resulting mixture was subjected to reaction under stirring at 350 rpm for 4.0 hours. Subsequently, 0.6 mol of 4-vinylbenzyl chloride was added thereto under stirring, and the reaction was continued under stirring for 8.0 hours. The reaction was then terminated, acetonitrile therein was evaporated, and 500 mL of water and 500 mL of toluene were then added thereto. A resulting system was layered, and a resulting organic phase was washed with 500 mL of a saturated ammonium chloride solution, and washed with water to neutral. Toluene therein was evaporated under reduced-pressure, and a resulting solid was subjected to pulping (30° C.) with 500 mL of methanol twice. A resulting system was filtered to obtain a solid, and the solid was subjected to recrystallization twice with toluene. A resulting solid was then vacuum-dried at 80° C. to a constant weight, obtaining 106.6 g of 9-(2-vinylbenzyl)-9′-(4-vinylbenzyl)-9H-fluorene (o,p-BVBF) as a white crystal, having a purity (HPLC) of 98.7%, with a yield of 53.5%.

### Example 8

20 g of the o,p-BVBF prepared in Example 1 and 0.1 g of T311 peroxide initiator were dissolved in 50 g of toluene. After solvent removal under reduced pressure, a thermosetting resin was prepared by a melt casting method (thermal curing conditions: a temperature of 220° C., a pressure of 70-80 mmHg, 100 minutes). A glass transition temperature was measured. The prepared thermosetting resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

### Example 9

500 mL of acetonitrile, 1.4 mol of potassium tert-butoxide, 30 g of PEG-400, and 0.5 g of polymerization inhibitor 701 were added to a reaction flask. 0.6 mol of fluorene and 1.4 mol of 4-vinylbenzyl chloride (HPLC grade, 99%) were added thereto under stirring and a nitrogen flow. A resulting mixture was subjected to reaction at 30-35° C. under stirring at 350 rpm until HPLC analysis confirmed a fluorene content was less than 1 wt %. The reaction was terminated, acetonitrile therein was evaporated, and 500 mL of water and 500 mL of toluene were added thereto. A resulting mixed system was layered, and a resulting organic phase was separated, washed with saturated ammonium chloride solution, and then washed three times with water (500 mL of water each time) until neutral. Toluene was removed under reduced pressure, and a toluene/methanol mixed solvent (v/v=1:0.3) was added thereto. A resulting system was heated to complete dissolution, cooled at a rate of 0.5° C./min to 5° C. for constant-temperature crystallization, and filtered. Aresulting solid was dried at 90° C. to a constant weight, obtaining 9,9-bis(2-vinylbenzyl)-9H-fluorene (p,p-BVBF) as a white crystal, having a melting point of 118.5-120.0° C., and a HPLC purity of 99.5%.

20 g of 9,9-bis(4-vinylbenzyl)-9H-fluorene (p,p-BVBF) and 0.1 g of T311 peroxide initiator were dissolved in 50 g of toluene. After solvent removal under reduced pressure, a thermosetting hydrocarbon resin was prepared by a melt casting method (thermal curing conditions: a temperature of 220° C., a pressure of 70-80 mmHg, 100 minutes). A glass transition temperature was measured. The prepared thermosetting hydrocarbon resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

### Example 10

9,9-Bis(2-vinylbenzyl)-9H-fluorene (p,p-BVBF) was prepared as described in Example 9, obtaining a white crystal, having a melting point of 118.5-120.0° C. and a HPLC purity of 99.5%.

10 g of the 9-(2-vinylbenzyl)-9′-(4-vinylbenzyl)-9H-fluorene (o,p-BVBF) prepared in Example 1, 10 g of the p,p-BVBF, and 0.1 g of T311 peroxide initiator were dissolved in 50 g of toluene. After solvent removal under reduced pressure, a thermosetting hydrocarbon resin was prepared by a melt casting method (thermal curing conditions: a temperature of 220° C., a pressure of 70-80 mmHg, 100 minutes). A glass transition temperature was measured. The prepared thermosetting hydrocarbon resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

### Example 11

150 mL of methanol, 0.09 mol of N,N-di-tert-butylethylenediamine, and 0.07 mol of copper bromide were added to a 2 L four-necked flask, and stirred until dissolution. Oxygen was introduced and continuously bubbled, and a resulting mixture was subjected to reaction under stirring at a temperature controlled at 40-45° C. A mixed solution of 2,6-dimethylphenol (1.0 mol) and tetramethylbisphenol A (0.125 mol) in toluene/methanol (500 mL/150 mL) was added dropwise thereto. After complete addition, introducing oxygen and continuously bubbling were maintained, the reaction continued under stirring for 4 hours at the same temperature. After the reaction, a reaction system was neutralized to be neutral with 10% HCl (aq), and 50 mL of an aqueous EDTA-2Na (7 g, 0.04 mol) was added thereto. A resulting mixture was stirred for 30 min. 600 mL of methanol was then added thereto, and precipitation occurred. A precipitate was filtered and subjected to pulping three times with methanol (300 mL each time), followed by vacuum drying at 80° C. for 8 hours, obtaining 285.0 g of polyphenylene ether (XSPPO); Mn=1750, Mw=2030.

120 mL of toluene, 60 g of the polyphenylene oxide (SXPPO), 60 mL of 50% aqueous sodium hydroxide solution, 4 g (0.043 mol) of tetrabutylammonium bromide, 0.1 g of polymerization inhibitor 701, and 15 g (0.18 mol) of p-chloromethylstyrene were added to a 500 mL four-necked flask. A resulting mixture was stirred in a nitrogen atmosphere while being heated to 70° C., and subjected to reaction under stirring for 8 hours. A resulting reaction system was cooled to room temperature, and neutralized with 10% HCl (aq) to neutral. A resulting organic phase was washed three times with 200 mL of water. 800 mL of methanol was added thereto, and precipitation occurred. A precipitate was filtered, washed with methanol/water (80:20 w/w), and dried under vacuum at 80° C. for 8 hours, obtaining 82 g of a modified polyphenylene ether (XSmPPO); Mn=1860, Mw=2180. A structural formula of the XSmPPO is as follows:

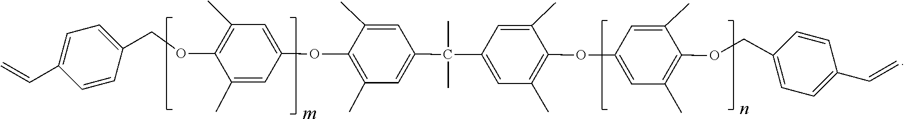

4 g (0.01 mol) of the 9-(2-vinylbenzyl)-9′-(4-vinylbenzyl)-9H-fluorene (o,p-BVBF) prepared in Example 1, 16 g of the XSmPPO, and 0.1 g of T311 peroxide initiator were dissolved in 50 g of toluene. After solvent removal under reduced pressure, a cross-linked resin was then prepared by a melt casting method (thermal curing conditions: a temperature of 220° C., a pressure of 70-80 mmHg, 100 minutes). A glass transition temperature was measured. The cross-linked resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

### Example 12

9,9-bis(4-vinylbenzyl)-9H-fluorene (p,p-BVBF) was prepared according to the method as described in Example 9.

A modified polyphenylene ether (XSmPPO) was prepared according to the method as described in Example 11;

4 g (0.01 mol) of the p,p-BVBF, 16 g of the XSmPPO, and 0.1 g of T311 peroxide initiator were dissolved in 50 g of toluene. After solvent removal under reduced pressure, a cross-linked resin was then prepared by a melt casting method (thermal curing conditions: a temperature of 220° C., a pressure of 70-80 mmHg, 100 minutes). A glass transition temperature was measured. The cross-linked resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

### Example 13

9,9-bis(4-vinylbenzyl)-9H-fluorene (p,p-BVBF) was prepared according to the method as described in Example 9.

A modified polyphenylene ether (XSmPPO) was prepared according to the method as described in Example 11.

2 g (0.005 mol) of 9-(2-vinylbenzyl)-9′-(4-vinylbenzyl)-9H-fluorene (o,p-BVBF) prepared in Example 1, 2 g (0.005 mol) of the p,p-BVBF, 16 g of the XSmPPO, and 0.1 g of T311 peroxide initiator were dissolved in 50 g of toluene. After solvent removal under reduced pressure, a cross-linked resin was then prepared by a melt casting method (thermal curing conditions: a temperature of 220° C., a pressure of 70-80 mmHg, 100 minutes). A glass transition temperature was measured. The cross-linked resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

### Comparative Example 1

20 g of 1,2-bis(4-vinylphenyl)ethane (BVPE) and 0.1 g of T311 peroxide initiator were dissolved in 50 g of toluene. The solvent was removed under reduced pressure, and a thermosetting hydrocarbon resin was then prepared by a melt casting method (thermal curing conditions: a temperature of 220° C., a pressure of 70-80 mmHg, 100 minutes). A glass transition temperature was measured. The thermosetting hydrocarbon resin was molded into 80 mm×80 mm×0.4 mm resin sheets, and a dielectric constant and a dielectric dissipation factor of a sample at a frequency of 10 GHz were measured using an Agilent N5230A vector network analyzer (Agilent Technologies, USA). Results are listed in Table 4.

### Comparative Example 2

A modified polyphenylene ether (XSmPPO) was prepared according to the method as described in Example 11.

20 g of the modified polyphenylene ether (XSmPPO) and 0.1 g of T311 peroxide initiator were dissolved in 50 g of toluene; and the solvent was removed under reduced pressure. A thermosetting resin was then prepared by a melt casting method (thermal curing conditions: a temperature of 220° C., a pressure of 70-80 mmHg, 100 minutes). A glass transition temperature was measured. The thermosetting resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

### Comparative Example 3

A modified polyphenylene ether (XSmPPO) was prepared according to the method as described in Example 11.

2.34 g (0.01 mol) of 1,2-bis(4-vinylphenyl)ethane (BVPE), 17.66 g of the modified polyphenylene ether (XSmPPO), and 0.1 g of T311 peroxide initiator were dissolved in 50 g of toluene, and the solvent was removed under reduced pressure. A cross-linked resin was then prepared by a melt casting method (thermal curing conditions: a temperature of 220° C., a pressure of 70-80 mmHg, 100 minutes). A glass transition temperature was measured. The cross-linked resin was molded into 80 mm×80 mm×0.4 mm sheets, and a dielectric constant and a dielectric dissipation factor of a sample at a frequency of 10 GHz were measured using an Agilent N5230A vector network analyzer (Agilent Technologies, USA). Results are listed in Table 4.

TABLE 4
Performance parameters of thermosetting resins prepared
in Examples 8-13 and Comparative Examples 1-3

| | | | | | | Performance | | |
|---|---|---|---|---|---|---|---|---|
| | Amount of raw materials (g) | | | | | Dk | Df | |
| | o,p- | p,p- | | | | (10 | (10 | Tg |
| Examples | BVBF | BVBF | BVPE | XSmPPO | Initiator | GH) | GH) | (° C.) |
| Example 8 | 20 | 0 | 0 | 0 | 0.1 | 2.8 | 0.00030 | 360 |
| Example 9 | 0 | 20 | 0 | 0 | 0.1 | 2.8 | 0.00047 | 320 |
| Example 10 | 10 | 10 | 0 | 0 | 0.1 | 2.8 | 0.00042 | 352 |
| Example 11 | 4 g | 0 | 0 | 16 | 0.1 | 2.8 | 0.00060 | 285 |
| | (0.01 mol) | | | | | | | |
| Example 12 | 0 | 4 g | 0 | 16 | 0.1 | 2.8 | 0.00070 | 256 |
| | | (0.01 mol) | | | | | | |
| Example 13 | 2 g | 2 g | 0 | 16 | 0.1 | 2.8 | 0.00065 | 261 |
| | (0.005 mol) | (0.005 mol) | | | | | | |
| Comparative | 0 | 0 | 20 | 0 | 0.1 | 2.8 | 0.00080 | 312 |
| Example 1 | | | | | | | | |
| Comparative | 0 | 0 | 0 | 20 | 0.1 | 3.2 | 0.00250 | 222 |
| Example 2 | | | | | | | | |
| Comparative | 0 | 0 | 2.34 g | 17.66 | 0.1 | 3.2 | 0.00120 | 230 |
| Example 3 | | | (0.01 mol) | | | | | |

In Table 4, Dk refers to dielectric constant, Df refers to dielectric dissipation factor, and Tg refers to glass transition temperature.

It can be seen from Table 4 that the thermosetting hydrocarbon resin prepared from 9,9-bis(vinylbenzyl)-9H-fluorene has a very low dielectric dissipation factor and a relatively high glass transition temperature (Examples 8, 9, and 10). The thermosetting hydrocarbon resin prepared from 9-(2-vinylbenzyl)-9′-(4-vinylbenzyl)-9H-fluorene (o,p-BVBF) has the lowest dielectric dissipation factor and the highest glass transition temperature (Example 8). The cross-linked resins obtained from the 9-(2-vinylbenzyl)-9′-(4-vinylbenzyl)-9H-fluorene (o,p-BVBF) (Example 11), the 9,9-bis(4-vinylbenzyl)-9H-fluorene (p,p-BVBF) (Example 12), or a 1:1 mixture of the two (Example 13) and vinyl-terminated modified polyphenylene ether show a significantly decreased dielectric constant, a largely decreased dielectric dissipation factor, and a significantly increased glass transition temperature, compared with the cross-linked resin obtained from the commonly used cross-linking agent 1,2-bis(4-vinylphenyl)ethane (BVPE) and the vinyl-terminated modified polyphenylene ether (Comparative Example 3). Moreover, a higher weight proportion of o,p-BVBF used as a cross-linking agent results in a lower dielectric dissipation factor and a higher glass transition temperature. Therefore, the thermosetting hydrocarbon resin and thermosetting cross-linked resin with the modified polyphenylene ether according to the present disclosure have excellent comprehensive performance and could be used as resin materials for high-frequency and high-speed printed circuit boards.

Although the above embodiments have provided a detailed description of the present disclosure, they are only a part of embodiments and not all of them. One person could also obtain other embodiments based on these embodiments without creativity, which all fall within the scope of the present disclosure.

What is claimed is:

1. An asymmetric bis(vinylbenzyl) fluorene, wherein the asymmetric bis(vinylbenzyl) fluorene is 9-(2-vinylbenzyl)-9′-(4-vinylbenzyl)-9H-fluorene having a structure shown in formula 1: 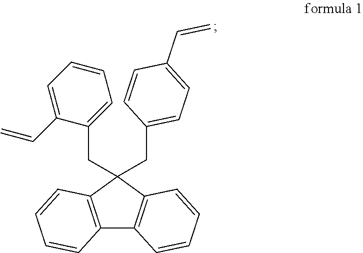 [SMILES: C=Cc1ccc(CC2(Cc3ccccc3C=C)c3ccccc3-c3ccccc32)cc1]
and
the 9-(2-vinylbenzyl)-9′-(4-vinylbenzyl)-9H-fluorene exhibits a thermal analysis melting endothermic peak in a temperature range of 169-175° C., and has a monoclinic crystal system and a space group of P2₁/c.

2. A method for preparing the asymmetric bis(vinylbenzyl) fluorene as claimed in claim 1, comprising:
mixing fluorene, a basic reagent, a polymerization inhibitor, and a polar aprotic solvent to obtain a fluorene-containing mixed system; and
dropwise adding 2-vinylbenzyl chloride and 4-vinylbenzyl chloride sequentially to the fluorene-containing mixed system, or dropwise adding a mixed solution of 2-vinylbenzyl chloride and 4-vinylbenzyl chloride to the fluorene-containing mixed system, and subjecting a resulting mixture to nucleophilic substitution reaction to obtain the asymmetric bis(vinylbenzyl) fluorene.

3. The method as claimed in claim 2, wherein the basic reagent comprises at least one selected from the group consisting of an alkali metal hydroxide, an alkali metal alkoxide, sodium hydride, and potassium hydride;
the polymerization inhibitor comprises at least one selected from the group consisting of nitromethane, nitrobenzene, o-nitrophenol, phenothiazine, 2-phenylnaphthylamine, hydroquinone, catechol, p-methoxyphenol, 2,6-di-tert-butyl-p-cresol, p-tert-butyl catechol, 2,2,6,6-tetramethylpiperidine oxynitride, and 2,2,6,6-tetramethyl-4-hydroxypiperidine oxynitride;
the polar aprotic solvent comprises one selected from the group consisting of dimethyl sulfoxide, sulfolane, N,N-dimethylformamide, N,N-diethylformamide, acetonitrile, and hexamethylphosphoramide;
a molar ratio of the fluorene to the basic reagent is in a range of 1:1.8 to 1:5;
a molar ratio of the 2-vinylbenzyl chloride to the 4-vinylbenzyl chloride is in a range of 2-6:4-8; and
a ratio of a total amount in moles of the 2-vinylbenzyl chloride and the 4-vinylbenzyl chloride to an amount in moles of the fluorene is in a range of 1.8:1 to 2.2:1.

4. The method as claimed in claim 2, wherein the nucleophilic substitution reaction is conducted at a temperature of 20-50° C. for 2-8 hours.

5. The method as claimed in claim 3, wherein the nucleophilic substitution reaction is conducted at a temperature of 20-50° C. for 2-8 hours.

6. A method for preparing the asymmetric bis(vinylbenzyl) fluorene as claimed in claim 1, comprising:
mixing fluorene, a basic reagent, a polymerization inhibitor, a phase transfer catalyst, and a solvent to obtain a fluorene-containing phase transfer catalytic system; and
dropwise adding 2-vinylbenzyl chloride and 4-vinylbenzyl chloride sequentially to the fluorene-containing phase transfer catalytic system, or dropwise adding a mixed solution of 2-vinylbenzyl chloride and 4-vinylbenzyl chloride to the fluorene-containing phase transfer catalytic system, and subjecting a resulting mixture to nucleophilic substitution reaction under phase transfer catalysis conditions to obtain the asymmetric bis(vinylbenzyl) fluorene.

7. The method as claimed in claim 6, wherein the basic reagent comprises at least one selected from the group consisting of an alkali metal hydroxide and an alkali metal alkoxide;
the polymerization inhibitor comprises at least one selected from the group consisting of nitromethane, nitrobenzene, o-nitrophenol, phenothiazine, 2-phenylnaphthylamine, hydroquinone, catechol, p-methoxyphenol, 2,6-di-tert-butyl-p-cresol, p-tert-butyl catechol, 2,2,6,6-tetramethylpiperidine oxynitride, and 2,2,6,6-tetramethyl-4-hydroxypiperidine oxynitride;
the phase transfer catalyst comprises at least one selected from the group consisting of a quaternary ammonium salt, a quaternary phosphonium salt, and polyethylene glycol;
a mass of the phase transfer catalyst is 5-25% of a mass of the fluorene;
a molar ratio of the fluorene to the basic reagent is in a range of 1:1.8 to 1:5;
a molar ratio of the 2-vinylbenzyl chloride to the 4-vinylbenzyl chloride is in a range of 2-6:4-8; and
a ratio of a total amount in moles of the 2-vinylbenzyl chloride and the 4-vinylbenzyl chloride to an amount in moles of the fluorene is in a range of 1.8:1 to 2.2:1.

8. The method as claimed in claim 6, wherein the nucleophilic substitution reaction under the phase transfer catalysis conditions is conducted at a temperature of 25-75° C. for 8-18 hours.

9. The method as claimed in claim 7, wherein the nucleophilic substitution reaction under the phase transfer catalysis conditions is conducted at a temperature of 25-75° C. for 8-18 hours.

10. A thermosetting resin, comprising at least one selected from the group consisting of a thermosetting hydrocarbon resin and a thermosetting cross-linked resin, wherein
the thermosetting hydrocarbon resin comprises at least one selected from the group consisting of a thermosetting hydrocarbon resin prepared by using 9-(2-vinylbenzyl)-9′-(4-vinylbenzyl)-9H-fluorene as a raw material and a thermosetting hydrocarbon resin prepared by using 9-(2-vinylbenzyl)-9′-(4-vinylbenzyl)-9H-fluorene and 9,9′-bis(4-vinylbenzyl)-9H-fluorene as raw materials;
the thermosetting cross-linked resin comprises a thermosetting cross-linked resin prepared by using a cross-linking agent and an alkenyl-terminated polyphenylene ether as raw materials, the cross-linking agent comprises at least one selected from the group consisting of 9-(2-vinylbenzyl)-9′-(4-vinylbenzyl)-9H-fluorene and 9,9′-bis(4-vinylbenzyl)-9H-fluorene, and the alkenyl-terminated polyphenylene ether has a structure selected from the group consisting of formula 2, formula 3, formula 4, and formula 5, 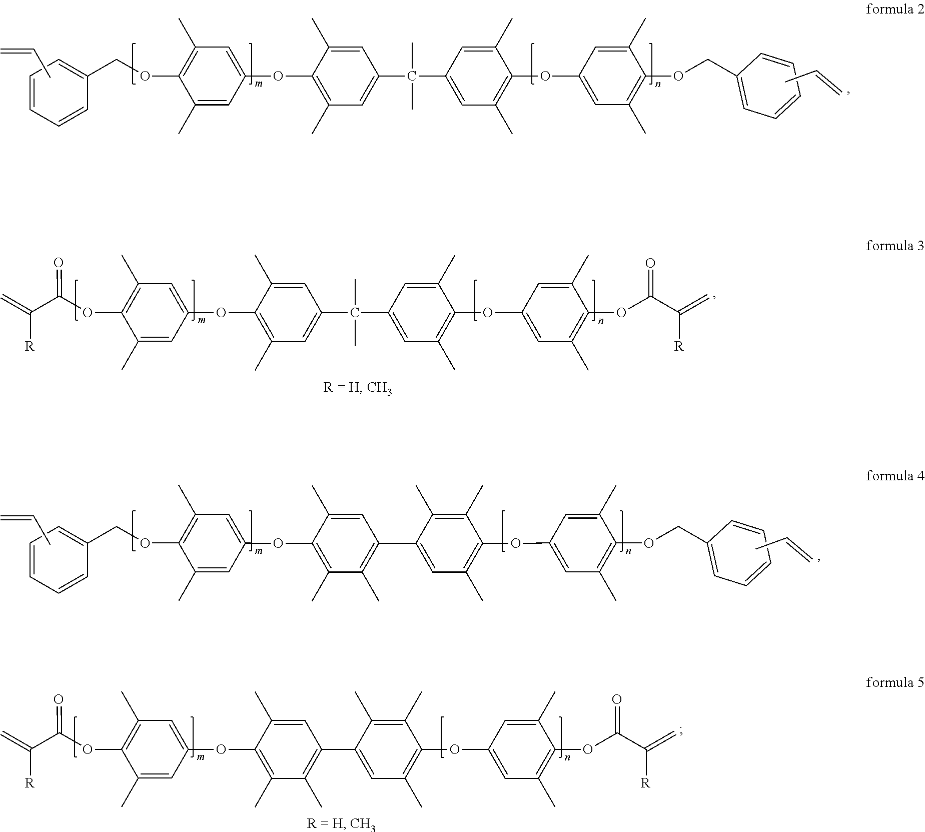
and
the 9-(2-vinylbenzyl)-9′-(4-vinylbenzyl)-9H-fluorene is the asymmetric bis(vinylbenzyl) fluorene as claimed in claim 1.

11. A method for preparing the thermosetting resin as claimed in claim 10, comprising:
dissolving the raw material(s) and an initiator in toluene to obtain a mixed solution; and
removing the toluene from the mixed solution, and then conducting melting thermal curing to obtain the thermosetting resin.
